(19) United States
(12) Patent Application Publication
(10) Pub. No.: US 2024/0183785 A1
(43) Pub. Date: Jun. 6, 2024

(54) Laser-Based Fast Micromanufacturing of Test Device for Rapid Detection of Pathogens

(71) Applicant: Carnegie Mellon University, Pittsburgh, PA (US)

(72) Inventors: Rahul Panat, Sewickley, PA (US); Azahar Ali, Pittsburgh, PA (US)

(21) Appl. No.: 18/284,993

(22) PCT Filed: Mar. 31, 2022
(86) PCT No.: PCT/US22/22762

§ 371 Date: Sep. 29, 2023

Related U.S. Application Data
(60) Provisional application No. 63/168,588, filed on Mar. 31, 2021.

(51) Int. Cl. G01N 21/77; G01N 21/35; G01N 21/3577; G01N 21/47; G01N 33/543

(52) U.S. Cl. CPC G01N 21/77; G01N 21/3577; G01N 21/4788; G01N 33/54373; G01N 2021/3595; G01N 2021/7779

(57) ABSTRACT

A photonic crystal for detection of an analyte includes: a first layer including a first material with a first refractive index; a second layer over the first layer and including a second material with a second refractive index that is higher than the first refractive index; where the second layer includes a hole, the hole including: a first diameter from an outer surface of the second layer to a first hole depth; a second diameter from the first hole depth to a second hole depth; where the first diameter is larger than the second diameter; and a member of a binding pair with the analyte linked to a surface of the hole.

### CROSS REFERENCE TO RELATED APPLICATIONS

This application claims the benefit of U.S. Provisional Application, No. 63/168,588, filed Mar. 31, 2021, which is incorporated herein by reference in its entirety.

### BACKGROUND OF THE INVENTION

Point-of-care (PoC) testing devices that can rapidly identify pathogens are critical in healthcare emergencies such as viral or bacterial pandemics. Further, fast manufacture of such devices results in lower cost, which leads to increased testing, lower community transmission, and a significantly reduced loss of life during a pandemic. In order to effectively lower community pathogen transmission, numerous PoC testing devices are needed so that fast and efficient testing is available to the community.

There is a need for a PoC testing device that can be rapidly manufactured and that can be used to quickly identify pathogens to stop community transmission of emerging pathogens.

### SUMMARY

According to an aspect or embodiment, a photonic crystal for detection of an analyte is provided, comprising: a first layer comprising a first material with a first refractive index; a second layer over the first layer and comprising a second material with a second refractive index that is higher than the first refractive index; wherein the second layer comprises a hole, the hole comprising: a first diameter from an outer surface of the second layer to a first hole depth; a second diameter from the first hole depth to a second hole depth; wherein the first diameter is larger than the second diameter; and a member of a binding pair with the analyte linked to a surface of the hole.

According to another aspect or embodiment, a method of manufacturing a photonic crystal is provided, comprising: providing a first layer comprising a first material with a first refractive index; depositing a second layer comprising a second material with a second refractive index that is higher than the first refractive index over the first layer; machining a hole in the second layer with a laser, comprising: machining the hole with a first diameter from an outer surface of the second layer to a first hole depth; machining the hole with a second diameter from the first hole depth to a second hole depth; wherein the first diameter is larger than the second diameter; and linking a member of a binding pair with an analyte linked to a surface of the hole.

According to another aspect or embodiment, a method of detecting pathogens in a fluid sample is provided, comprising: providing a detection device comprising: a substrate; a device body positioned over the substrate and comprising an inlet channel and an outlet channel; a photonic crystal in fluid communication with the inlet channel and the outlet channel, the photonic crystal comprising: a first layer comprising a first material with a first refractive index; a second layer over the first layer and comprising a second material with a second refractive index that is higher than the first refractive index; wherein the second layer comprises a hole, the hole comprising: a first diameter from an outer surface of the second layer to a first hole depth; and a second diameter from the first hole depth to a second hole depth; wherein the first diameter is larger than the second diameter; and a member of a binding pair with the analyte linked to a surface of the hole; introducing the fluid sample into the inlet channel; passing the fluid sample over the photonic crystal; exposing the fluid sample and photonic crystal to light from a light source; detecting the light that passes through and/or is reflected from the photonic crystal with a light detector; and passing the fluid sample to the outlet channel.

Other non-limiting embodiments or aspects are set forth in the following illustrative and exemplary numbered clauses:

Clause 1: A photonic crystal for detection of an analyte, comprising: a first layer comprising a first material with a first refractive index; a second layer over the first layer and comprising a second material with a second refractive index that is higher than the first refractive index; wherein the second layer comprises a hole, the hole comprising: a first diameter from an outer surface of the second layer to a first hole depth; a second diameter from the first hole depth to a second hole depth; wherein the first diameter is larger than the second diameter; and a member of a binding pair with the analyte linked to a surface of the hole.

Clause 2: The photonic crystal of clause 1, wherein the first material comprises silicon dioxide, nitride, indium phosphide, lithium niobite, sapphire, or a combination thereof.

Clause 3: The photonic crystal of clause 1 or clause 2, wherein the first material comprises silicon dioxide.

Clause 4: The photonic crystal of any of clauses 1-3, further comprising a third layer under the first layer and comprising a third material with a third refractive index, wherein the third refractive index is higher than the first refractive index.

Clause 5: The photonic crystal of clause 4, wherein the second material and the third material each independently comprise silicon, gallium arsenide, gallium nitride, silicon carbide, indium gallium arsenide, or a combination thereof.

Clause 6: The photonic crystal of clause 5, wherein the second material and the third material comprise silicon.

Clause 7: The photonic crystal of any of clauses 1-6, wherein the second layer comprises a plurality of holes.

Clause 8: The photonic crystal of any of clauses 1-7, wherein the hole comprises a third diameter from the second hole depth to a third hole depth, wherein the second diameter is larger than the third diameter.

Clause 9: The photonic crystal of any of clauses 1-8, wherein the first diameter is between 0.5 μm and 5 μm.

Clause 10: The photonic crystal of any of clauses 1-9, wherein the second diameter is between 0.25 μm and 4 μm

Clause 11: The photonic crystal of any of clauses 1-10, wherein the first hole depth is between 0.1 μm and 1 μm.

Clause 12: The photonic crystal of any of clauses 1-11, wherein the second hole depth is between 0.25 μm and 2 μm.

Clause 13: The photonic crystal of any of clauses 1-12, wherein the second hole depth is the thickness of the second layer.

Clause 14: The photonic crystal of any of clauses 8-13, wherein the third diameter is between 0.1 μm and 3 μm.

Clause 15: The photonic crystal of any of clauses 8-14, wherein the third hole depth is between 0.5 μm to 3 μm.

Clause 16: The photonic crystal of any of clauses 8-15, wherein the third hole depth is the thickness of the second layer.

Clause 17: The photonic crystal of any of clauses 1-16, wherein the member of a binding pair with the analyte is an antibody.

Clause 18: The photonic crystal of any of clauses 1-17, wherein the analyte is a pathogen or an antibody specific to a pathogen.

Clause 19: The photonic crystal of any of clauses 1-18, comprising one or more additional holes and the holes are discretely-addressable in an array, wherein at least one of the one or more additional holes comprises a different member of a binding pair as compared to the member of a binding pair with the analyte bound to its surface, or no member of a binding pair bound to its surface.

Clause 20: A method of manufacturing a photonic crystal, comprising: providing a first layer comprising a first material with a first refractive index; depositing a second layer comprising a second material with a second refractive index that is higher than the first refractive index over the first layer; machining a hole in the second layer with a laser, comprising: machining the hole with a first diameter from an outer surface of the second layer to a first hole depth; machining the hole with a second diameter from the first hole depth to a second hole depth; wherein the first diameter is larger than the second diameter; and linking a member of a binding pair with an analyte linked to a surface of the hole.

Clause 21: The method of clause 20, wherein the laser is an excimer laser.

Clause 22: The method of clause 20 or clause 21, wherein the machining of the hole further comprises: machining the hole with a third diameter from the second hole depth to a third hole depth, wherein the second diameter is larger than the third diameter.

Clause 23: The method of any of clauses 20-22, further comprising: depositing an antibody or antibodies into the hole.

Clause 24: The method of any of clauses 20-23, further comprising machining a plurality of holes in the second layer.

Clause 25: A method of detecting pathogens in a fluid sample, comprising: providing a detection device comprising: a substrate; a device body positioned over the substrate and comprising an inlet channel and an outlet channel; a photonic crystal in fluid communication with the inlet channel and the outlet channel, the photonic crystal comprising: a first layer comprising a first material with a first refractive index; a second layer over the first layer and comprising a second material with a second refractive index that is higher than the first refractive index; wherein the second layer comprises a hole, the hole comprising: a first diameter from an outer surface of the second layer to a first hole depth; and a second diameter from the first hole depth to a second hole depth; wherein the first diameter is larger than the second diameter; and a member of a binding pair with the analyte linked to a surface of the hole; introducing the fluid sample into the inlet channel; passing the fluid sample over the photonic crystal; exposing the fluid sample and photonic crystal to light from a light source; detecting the light that passes through and/or is reflected from the photonic crystal with a light detector; and passing the fluid sample to the outlet channel.

Clause 26: The method of clause 25, wherein the fluid sample is blood, serum, saliva, and/or another bodily fluid.

Clause 27: The method of clause 25 or clause 26, wherein the light from the light source is monochromatic light having a wavelength between 380 nm and 100,000 nm.

Clause 28: The method of any of clauses 25-27, wherein the light source and the light detector is a Fourier-transform infrared spectroscopy device.

Clause 29: The method of any of clauses 25-28, wherein the photonic crystal comprises a plurality of holes.

Clause 30: The method of any of clauses 25-29, further comprising a plurality of photonic crystals between the inlet channel and the outlet channel, wherein each photonic crystal of the plurality of photonic crystals comprises a different antibody or antibodies.

### BRIEF DESCRIPTION OF THE DRAWINGS

FIG. 1A is a perspective view of a photonic crystal according to one aspect of the present invention;

FIG. 1B is a partial view of the photonic crystal of FIG. 1B;

FIG. 2 is a cross-sectional view of a photonic crystal according to another aspect of the present invention;

FIG. 3 is a cross-sectional view of a photonic crystal according to another aspect of the present invention;

FIG. 4 is a cross-sectional view of a photonic crystal according to another aspect of the present invention;

FIG. 5 is a schematic diagram of an antibody positioned in a hole according to another aspect of the present invention;

FIG. 6A is a perspective view of a detection device according to another aspect of the present invention;

FIG. 6B is a perspective view of a detection device according to another aspect of the present invention;

FIG. 7 is a schematic diagram of a detection device according to another aspect of the present invention.

### DETAILED DESCRIPTION

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of ranges is intended as a continuous range including every value between the minimum and maximum values. As used herein “a” and “an” refer to one or more.

As used herein, the term “comprising” is open-ended and may be synonymous with “including”, “containing”, or “characterized by”. The term “consisting essentially of” limits the scope of a claim to the specified materials or steps and those that do not materially affect the basic and novel characteristic(s) of the claimed invention. The term “consisting of” excludes any element, step, or ingredient not specified in the claim. As used herein, embodiments “comprising” one or more stated elements or steps also include, but are not limited to embodiments “consisting essentially of” and “consisting of” these stated elements or steps. The terms “visible region” or “visible light” refer to electromagnetic radiation having a wavelength in the range of 380 nm to 800 nm. The terms “infrared region” or “infrared radiation” refer to electromagnetic radiation having a wavelength in the range of greater than 800 nm to 100,000 nm. The terms “ultraviolet region” or “ultraviolet radiation” mean electromagnetic energy having a wavelength in the range of 300 nm to less than 380 nm.

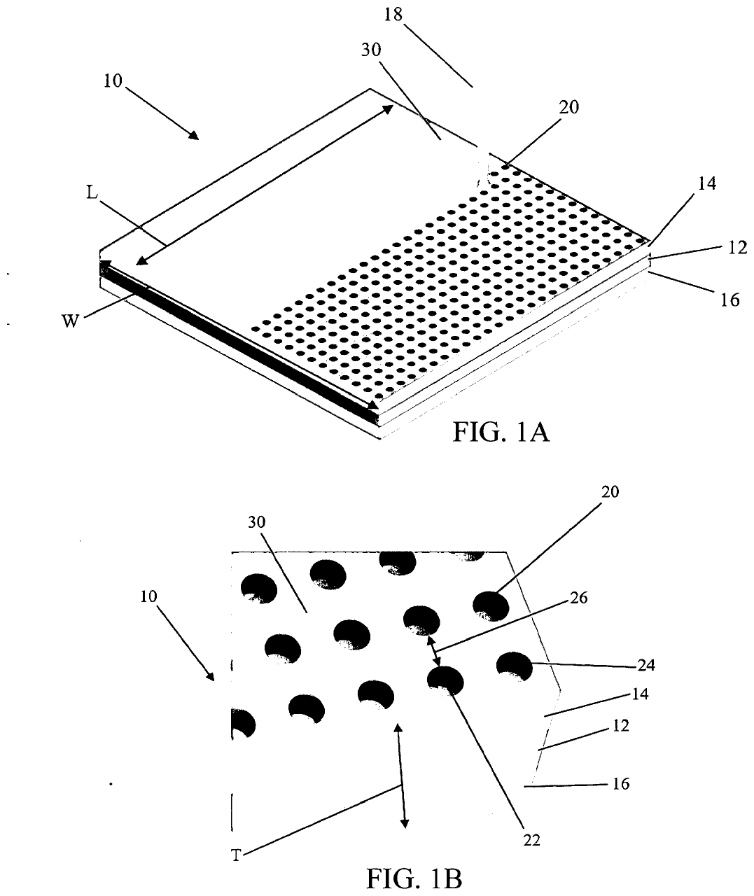

The present invention includes devices, methods, and products for the detecting on pathogens. Wave-guiding using periodic confinement of light can be achieved by features such as periodic arrays of holes of regular and/or irregular shapes and arrangements, with the same or different depths in a photonic crystal. Generally, a photonic crystal is a periodic structure in an optical medium, which creates certain optical dispersion properties. In the context of the present invention, optical transmission through a photonic crystal can respond directly to refractive index changes caused by the presence or absence of pathogens or antibodies on the crystal surface. Non-limiting embodiments of a photonic crystal are shown in FIGS. 1A-4. The photonic crystals described herein include holes, as described. In manufacture, distribution, and storage, the holes may comprise air or another fluid, such as a different gas, e.g., an inert gas, or a liquid, and may be referred to as “air holes”, if no liquid is present, but also may be distributed, stored, and used with a liquid within the holes.

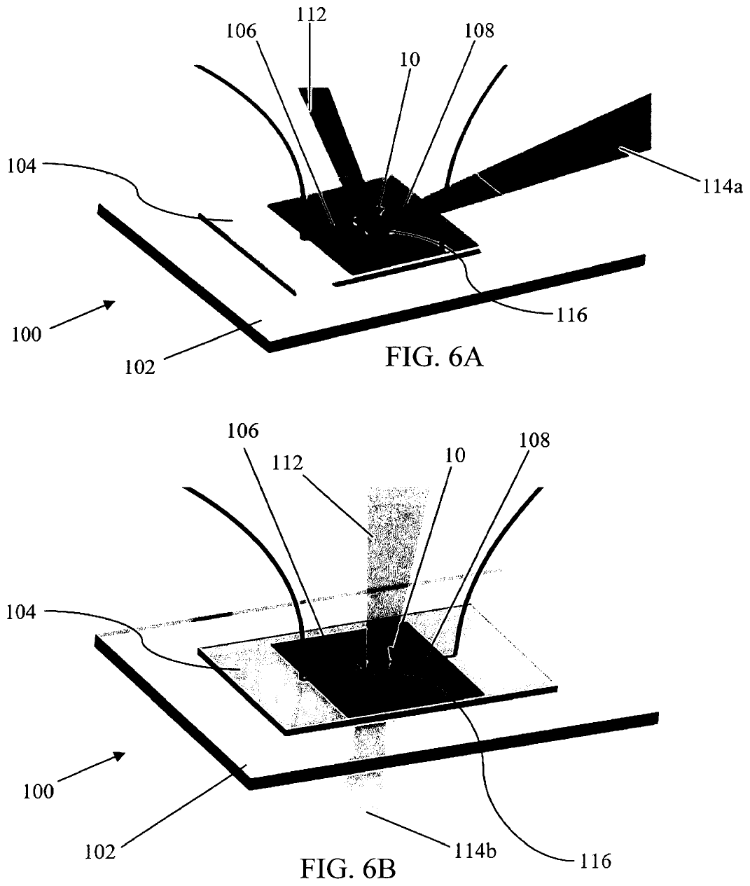

Referring to FIGS. 1A-4, in some non-limiting embodiments, a photonic crystal 10 for detection of pathogens is provided. The photonic crystal 10 may have a thickness T between 1 μm and 1,000 μm. In some non-limiting embodiments, the photonic crystal 10 may have a thickness T, as shown in FIG. 1B, that is the sum of the thicknesses of each of the layers of the photonic crystal 10. The photonic crystal 10 may have a shape that corresponds to an application of the photonic crystal 10. For example, the photonic crystal 10 may have a shape that corresponds to a shape of a detection device 100 that the photonic crystal 10 forms a part of, for example as depicted in FIGS. 6A and 6B. The photonic crystal 10 may include one or more layers. The one or more layers of the photonic crystal 10 may comprise various materials that are known in the art for use in photonic crystals. The photonic crystal 10 may have a length L between 100 μm and 10 mm and a width W between 100 μm and 10 mm. In some non-limiting embodiments, the photonic crystal 10 may be re-usable, such that the photonic crystal 10 may be used again after previously testing a fluid sample, for example after washing and/or eluting materials bound to the photonic crystal.

In some non-limiting embodiments, the photonic crystal 10 may comprise a first layer 12. The first layer 12 of the photonic crystal 10 may include a first material. The first material of the first layer 12 may have a first refractive index. The first refractive index may be less than 2. Examples of the first material of the first layer 12 include, but are not limited to, silicon dioxide, nitride, indium phosphide, lithium niobite, sapphire, or a combination thereof. For example, the first material of the first layer 12 may include silicon dioxide.

In some non-limiting embodiments, the first layer 12 of the photonic crystal may have a thickness such that light from a light source is transmitted through the first layer 12. A light source may emit electromagnetic radiation at any wavelength or spectral range, typically in the ultraviolet, visible, and infrared ranges. A light source may provide coherent light, as in the case of a laser, or non-coherent light, as with an incandescent or LED light source, which may be passed through, for example and without limitation; slits, holes, color filters, neutral filters, polarizing filters, diffraction gratings, lenses, or other optical devices, to provide a suitable light source for detection using the photonic crystals described herein. Suitable devices for providing a light source and/or detection devices are broadly known, such as a FTIR apparatus or spectrometers. In some non-limiting embodiments, the first layer 12 of the photonic crystal 10 may have a thickness such that light from a light source is reflected from the first layer 12 or is transmitted through the first layer 12. In some non-limiting embodiments, the first layer 12 of the photonic crystal 10 may have a thickness such that a portion of light from a light source is transmitted through the first layer 12, and another portion of the light from the light source is reflected from the first layer 12. The first layer 12 of the photonic crystal 10 may have a thickness between 0.1 μm and 10 μm, or between 0.25 μm and 5 μm, or between 0.5 μm and 3 μm.

In some non-limiting embodiments, a photonic crystal 10 may comprise a second layer 14. The second layer 14 of the photonic crystal 10 may be positioned over the first layer 12 of the photonic crystal 10. The second layer 14 of the photonic crystal 10 may include a second material. The second material of the second layer 14 may have a second refractive index. The second refractive index may be higher than the first refractive index. For example, the second refractive index may be greater than 2, or greater than 3, or greater than 4. Examples of the second material of the second layer 14 include, but are not limited to, silicon, gallium arsenide, gallium nitride, silicon carbide, indium gallium arsenide, or a combination thereof. For example, the second material of the second layer 14 may include silicon.

In some non-limiting embodiments, the second layer 14 of the photonic crystal 10 may have a thickness such that light from a light source is transmitted through the second layer 14. In some non-limiting embodiments, the second layer 14 may have a thickness such that light from a light source is reflected from the second layer 14. In some non-limiting embodiments, the second layer 14 of the photonic crystal 10 may have a thickness such that a portion of light from a light source is transmitted through the second layer 14, and another portion of the light from the light source is reflected from the second layer 14. The second layer 14 of the photonic crystal 10 may have a thickness between 0.05 μm and 5 μm, or between 0.1 μm and 3 μm, or between 0.1 μm and 2 μm.

In some non-limiting embodiments, the first layer 12 and/or the second layer 14 of the photonic crystal 10 may include other materials. For example, the first layer 12 and/or second layer 14 may include gold, graphene, cubic boron nitride (CBN), silver, and/or nickel.

In some non-limiting embodiments, the second layer 14 of the photonic crystal 10 may include a hole 20. For example, the second layer 14 of the photonic crystal 10 may include a plurality of holes 20. The hole 20, such as the plurality of holes 20, may extend from an outer surface 30 of the second layer 14 into the second layer 14. The hole 20, such as the plurality of holes 20, may include various shapes as the cross-section of the hole 20, such as, but not limited to, a circle, oval, square, rectangle, and/or the like. In some non-limiting embodiments, the photonic crystal 10 may include a plurality of holes 20 with a spacing 26 between the holes 20 between 1 μm and 5 μm. In some non-limiting embodiments, the hole 20, such as the plurality of holes 20, may have a diameter 22 between 500 nm to 100 μm. In some non-limiting embodiments, the hole 20, such as the plurality of holes 20, may have a depth 24 between 10 nm to 100 μm.

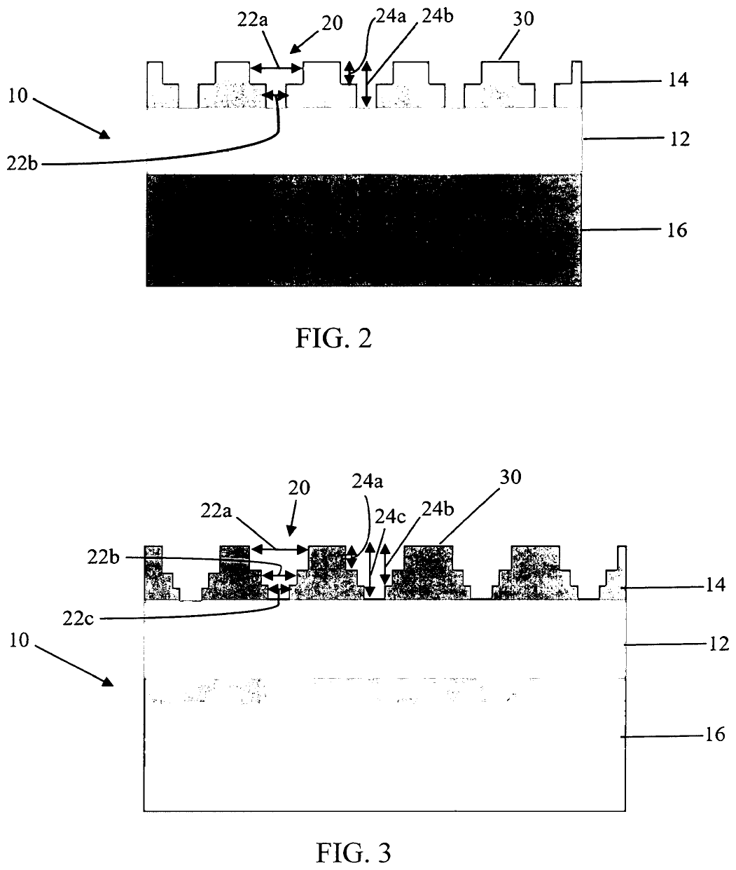

Referring to FIGS. 2 and 3, the hole 20, such as the plurality of holes 20, may be a stepped hole 20. As used herein, a “stepped” hole refers to a hole that includes more than one diameter throughout the depth of the hole. In some non-limiting embodiments, the hole 20, such as the plurality of holes 20, may include a first diameter 22a. The first diameter 22a may span from the outer surface 30 of the second layer 14 to a first hole depth 24a into the second layer 14. The first diameter 22a of the hole 20 may be between 0.5 μm and 5 μm, or between 0.5 μm and 4 μm, or between 0.5 μm and 3 μm, or between 1 μm and 3 μm. The first hole depth 24a, from the outer surface 30 of the second layer 14, may be between 0.1 μm and 2 μm, or between 0.1 μm and 1 μm, or between 0.25 μm and 0.75 μm.

In some non-limiting embodiments, the hole 20, such as the plurality of holes 20, may include a second diameter 22b. The second diameter 22b may span from the first hole depth 24a to a second hole depth 24b into the second layer 14. The first diameter 22a may be larger than the second diameter 22b. The second diameter 22b of the hole 20 may be between 0.25 μm and 4 μm, or between 0.25 μm and 3 μm, or between 0.5 μm and 3 μm, or between 1 μm and 2 μm. The second hole depth 24b, from the outer surface 30 of the second layer 14, may be between 0.25 μm and 2 μm, or between 0.25 μm and 1.5 μm, or between 0.25 μm and 1.25 μm, or between 0.5 μm and 1 μm. In some non-limiting embodiments, the second hole depth 24b may be equal to the thickness of the second layer 14. In such an example, the second hole depth 24b may expose a surface of the first layer 12 that is positioned under the second layer 14.

Referring to FIG. 3, in some non-limiting embodiments, the hole 20, such as the plurality of holes 20, may include a third diameter 22c. The third diameter 22c may span from the second hole depth 24b to a third hole depth 24c into the second layer 14. The second diameter 22b may be larger than the third diameter 22c. The third diameter 22c of the hole 20 may be between 0.1 μm and 3 μm, or between 0.1 μm and 2 μm, or between 0.5 μm and 1.5 μm. The third hole depth 24c, from the outer surface 30 of the second layer 14, may be between 0.5 μm and 3 μm, or between 0.5 μm and 2 μm, or between 0.75 μm and 1.5 μm. In some non-limiting embodiments, the third hole depth 24c may be equal to the thickness of the second layer 14. In such an example, the third hole depth 24c may expose a surface of the first layer 12 that is positioned under the second layer 14.

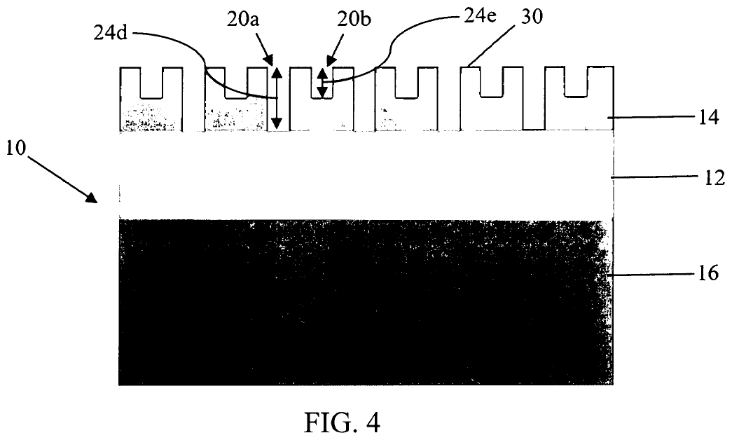

In some non-limiting embodiments, the photonic crystal 10 may include a plurality of holes 20. In some non-limiting embodiments, each hole 20 of the plurality of holes 20 may be different. For example, each hole 20 of the plurality of holes 20 may have a different first diameter 22a, second diameter 22b, third diameter 22c, first hole depth 24a, second hole depth 24b, and/or third hole depth 24c. In such an example, each hole 20 of the plurality of holes 20 may independently have a different first diameter 22a, second diameter 22b, third diameter 22c, first hole depth 24a, second hole depth 24b, and/or third hole depth 24c as previously described. For example, as shown in FIG. 4, a first hole 20a may have a depth 24d, from the outer surface 30 of the second layer 14, that is larger than the depth 24e of a second hole 20b. Holes 20 with varying depths and/or with different diameters will allow multiple peaks in the reflected or transmitted spectrum, which may lead to the detection of pathogens with a high-Q resonance; resulting in high sensitivity, high accuracy, and a lower limit of detection.

Any suitable light sensing device, such as CMOS or CCD sensors may be utilized to detect and produce a digital image or representation of light from the photonic crystal and transmit the data to a suitable processor with suitable instructions to collect, store, analyze, output, and otherwise handle and represent information received in a useful form, for example, in a spectrometer.

A member of a binding pair, that is a binding reagent or binding partner may be attached covalently to a surface of the photonic crystal 10 (e.g., linked or surface bound) within one or more of the holes 20, 20a or 20b. In use, and in context of the present disclosure, a liquid sample that may comprise an analyte that is a member of a binding pair with the linked or surface-bound member of the binding pair, may be contacted with the linked or surface-bound member of the binding pair, such that when the photonic crystal 10 is illuminated by a suitable light source, a detectable difference in a property of the light, such as light transmission, refraction, and/or reflection, is seen when the analyte is bound to the binding pair member linked to the photonic crystal 10 as compared to when no analyte is bound to the binding pair member. This effect may be quantitative in that differences in the amount of the analyte bound to the binding pair member of the photonic crystal may produce detectably-different differences in a property of the light. The analyte may be any substance that may bind specifically to its binding partner affixed to the photonic crystal 10, such as a pathogenic organism (pathogen, e.g., a virus particle or bacterium), a nucleic acid, a protein, a carbohydrate, a pharmaceutical active ingredient, a dye, a small molecule, a cytokine or a growth factor, or any other relevant analyte. The analyte is not limited to medically or pharmaceutically-relevant analytes and samples, but may include other samples, such as, for example and without limitation: water or wastewater samples, food samples, or soil samples.

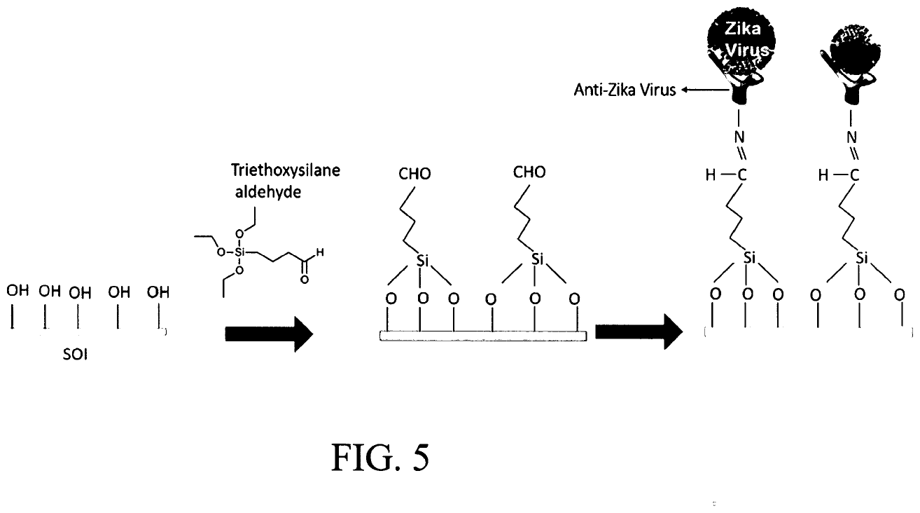

Referring to FIG. 5, in some non-limiting embodiments, an antibody or antibodies may be positioned in the hole 20, such as the plurality of holes 20. In FIG. 5, anti-Zika virus antibody is shown as a non-limiting example of an antibody that may be positioned in the hole 20. However, anti-Zika virus antibody is merely shown as an example, and can be replaced/combined with any antibody or antibodies, or any member of a binding pair, e.g., as described herein. The antibody or antibodies may be specific to a pathogen, or pathogens, that is/are to be detected by the photonic crystal 10. For example, in FIG. 5, anti-Zika virus is used in order to detect the presence of Zika virus in a sample. In such an example, when a fluid sample comprising a pathogen is passed over the hole 20, such as the plurality of holes 20, the pathogen may bond to the antibody or antibodies positioned in the hole 20, such as the plurality of holes 20. The antibody or antibodies may be bonded to the second layer 14 within the hole 20, and/or may be bonded to the first layer 12 if the depth 24 of the hole 20 exposes a surface of the first layer 12.

Antibodies are merely exemplary of binding reagents, that are members of a binding pair useful for detection of an analyte, such as a compound or a molecule on an organism, such as a virus particle. Use of antibodies and similar affinity binding reagents are broadly-known, and suitable binding pairs and members of binding pairs, e.g., binding partners, for detection using the methods and devices described herein may be identified readily by one of ordinary skill. Association of one molecule with another may be covalent or non-covalent. By attaching or linking one moiety to another, it is meant the linkage is covalent, as in, for example, polymerization, cross-linking, click chemistry reactions, or linking reactions using linkers. Complexing two molecules refers to a non-covalent association, such as by Van der Waals forces, hydrogen bonding, pi stacking, or ionic interactions, and may include specific binding reactions, such as in antibody-antigen binding, lectin-carbohydrate binding, or hybridization of two complementary oligonucleotides, nucleic acids, and/or nucleic acid analogs. In one example, a polypeptide sequence, e.g. an epitope, present on a protein of a virus particle, may bind specifically to an antibody.

In the context of recognition reagents, the term “ligand” refers to a binding moiety for a specific target, namely, its binding partner. Collectively the ligand and its binding partner are termed a binding pair. A binding partner can be a cognate receptor, a protein, a small molecule, a hapten, or any other relevant molecule, such as an affibody or another paratope-containing molecule. One common, and non-limiting example of a binding pair is streptavidin/avidin and biotin. The term “antibody” refers to an immunoglobulin, derivatives thereof which maintain specific binding ability, and proteins having a binding domain which is homologous or largely homologous to an immunoglobulin binding domain. As such, the antibody operates as a ligand for its cognate antigen, which can be virtually any type of molecule. Antibody mimetics are not antibodies, but comprise binding moieties or structures, e.g., paratopes, and include, for example, and without limitation: an affibody, an aptamer, an affilin, an affimer, an affitin, an alphabody, an aticalin, an avimer, a DARPin, a funomer, a Kunitz domain peptide, a monobody, a nanoclamp, or other engineered protein ligands, e.g., comprising a paratope targeting any suitable epitope present in a sample.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, Fv, Fd, dsFv, scFv, diabody, triabody, tetrabody, di-scFv (dimeric single-chain variable fragment), bi-specific T-cell engager (BiTE), single-domain antibody (sdAb), or antibody binding domain fragments. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids. Antibodies may be monoclonal or polyclonal, where different antibodies directed to the same antigen may be linked at a single location on the photonic crystal.

Ligands also include other engineered binding reagents, such as affibodies and designed ankyrin repeat proteins (DARPins), that exploit the modular nature of repeat proteins (Forrer T, Stumpp M T, Binz H K, Pluckthun A: A novel strategy to design binding molecules harnessing the modular nature of repeat proteins, FEBS Lett 2003, 539: 2-6; Gebauer A, Skerra A: Engineered protein scaffolds as next-generation antibody therapeutics, Curr Opin Chem Biol 2009, 13:245-255), comprising, often as a single chain, one or more antigen-binding or epitope-binding sequences and at a minimum any other amino acid sequences needed to ensure appropriate specificity, delivery, and stability of the composition. As would be appreciated, the full scope of suitable binding pairs for use in the devices and methods provided herein would be appreciated by those of ordinary skill.

In the context of the present disclosure, one member of a binding pair may be affixed to the device by any suitable linkage. As a non-limiting example, virus particles may be covalently-linked to the device, for binding to, e.g., for detection of or quantification of anti-virus antibodies in a biological sample, such that binding events with an antibody binding partner are detected. Conversely, an antibody binding partner of an antigen or epitope present on or in a virus particle may be covalently-linked to the device, for binding to, e.g., for detection of or quantification of virus particles in a sample. As depicted in the examples and FIG. 5 (described in further detail below), and as an example of one method of linking a proteinaceous compound, such as an antibody, or other amine-containing members of a binding pair to a photonic crystal, the material may be linked to pendant hydroxyl groups of a silicon dioxide or other modified silicon substrate, using triethoxysilane aldehyde.

In some non-limiting embodiments, a linking molecule may be provided in order to bond the antibody or antibodies to the second layer 14, and/or to the first layer 12 if the depth 24 of the hole 20 exposes a surface of the first layer 12. For example, in FIG. 5, a linking molecule triethoxysilane aldehyde is provided to bind the antibody or antibodies to the second layer 14 and/or to the first layer 12 if the depth 24 of the hole 20 exposes a surface of the first layer 12. However, triethoxysilane aldehyde is merely shown as an example, and can be replaced/combined with any linking molecule listed herein. Alternatively, in another example, triethoxysilane may be used as a linking molecule, and combined with an additional crosslinker, such as glutaraldehyde to introduce aldehyde groups (—CHO) for bonding to the antibody or antibodies. For example, a linking molecule may be provided that corresponds to the available groups present on the member of the binding pair to be positioned within the hole 20. Linking groups are broadly-known in the arts (see, e.g., Udomsom S, et al. Novel Rapid Protein Coating Technique for Silicon Photonic Biosensor to Improve Surface Morphology and Increase Bioreceptor Density. Coatings. 2021; 11(5):595). Examples of additional linking molecules for linking members of binding pairs to a silicon-containing substrate, include, but are not limited to coronavirus, e.g., SARS-Co-2, anti-spike S1 antibodies, spike S1 proteins, anti-receptor binding domain (RBD) antibodies, RBD proteins, anti-N-protein antibodies, and N-protein.

In some non-limiting embodiments, an antigen, such as a pathogen or a protein or other ligand or antigen present on or in the pathogen, may be positioned in the hole 20 instead of an antibody. The pathogen may bind specifically to a binding partner, such as an antibody, or antibodies, to be detected by the photonic crystal 10. In such an example, when a fluid sample, such as a bodily fluid of a patient, such as blood, plasma, serum, saliva, urine, etc., comprising an antibody is passed over the hole 20, such as the plurality of holes 20, the antibody may bond to the pathogen or pathogens positioned in the hole 20, such as the plurality of holes 20. The pathogen or pathogens may be bonded to the second layer 14 within the hole 20, and/or may be bonded to the first layer 12 if the depth 24 of the hole 20 exposes a surface of the first layer 12. In some non-limiting embodiments, a linking molecule may be provided to bond the pathogen or pathogens to the second layer 14 within the hole 20, and/or bond to the first layer 12 if the depth 24 of the hole 20 exposes a surface of the first layer 12. The linking molecule may be any of the previously described linking molecules. Examples of pathogens that may be positioned in the hole 20 include, but are not limited to virus particles, or surface proteins of organisms, such as viral surface proteins, e.g., spike, hemagglutinin, neuraminidase, or bacterial or fungal surface proteins. Exemplary, but non-limiting, viral particles including flavivirus or coronavirus particles, or surface proteins thereof, such as zika virus, or envelope proteins thereof, or SARS-COV-2, or spike proteins thereof.

The photonic crystals described herein may comprise an array or multiple (two or more), discrete and individually-addressable holes, in which the same or different members of a binding pair are linked as described herein. One or more holes may comprise no members of a binding pair as a control. Likewise, two or more holes may comprise the same members of a binding pair, e.g. for repeatability, and/or different surface densities of the same members of a binding pair, e.g. for quantification of an analyte in a sample to be tested. Holes may be spaced on the photonic crystal so as to produce a pattern of light that is different when analyte is present, so as to produce various effects, such as diffraction or interference patterns that may be more readily detectable as compared to effects produced by single holes.

Referring back to FIGS. 1A-4, in some non-limiting embodiments, the photonic crystal 10 may include a third layer 16. The third layer 16 of the photonic crystal 10 may be positioned under the first layer 12 of the photonic crystal 10. In such an example, the photonic crystal 10 may include a third layer 16, a first layer 12 over the third layer 16, and a second layer 14 over the first layer 12, wherein the second layer 14 includes a hole 20, such as a plurality of holes 20. The third layer 16 of the photonic crystal 10 may include a third material. The third material of the third layer 16 may have a third refractive index. The third refractive index may be higher than the first refractive index. For example, the third refractive index may be greater than 2, or greater than 3, or greater than 4. Examples of the third material of the third layer 16 include, but are not limited to, silicon, gallium arsenide, gallium nitride, silicon carbide, indium gallium arsenide, or a combination thereof. For example, the third material of the third layer 16 may include silicon. In some non-limiting embodiments, the third material may be the same as the second material. In some non-limiting embodiments, the third material may be different from the second material.

In some non-limiting embodiments, the third layer 16 of the photonic crystal 10 may have a thickness such that light from a light source is transmitted through the third layer 16. In some non-limiting embodiments, the third layer 16 may have a thickness such that light from a light source is reflected from the third layer 16. In some non-limiting embodiments, the third layer 16 of the photonic crystal 10 may have a thickness such that a portion of light from a light source is transmitted through the third layer 16, and another portion of the light from the light source is reflected from the third layer 16. The third layer 16 of the photonic crystal 10 may have a thickness between 1 μm and 1,000 μm, or between 1 μm and 750 μm, or between 5 μm and 750 μm.

The present invention also relates to a method of manufacturing a photonic crystal 10. In some non-limiting embodiments, the method of manufacturing a photonic crystal 10 may include providing a first layer 12. The first layer 12 may be the same as the previously described first layer 12. The method of manufacturing a photonic crystal 10 may further include depositing a second layer 14 over the first layer 12. The second layer 14 may be the same as the previously described second layer 14.

In some non-limiting embodiments, the method of manufacturing a photonic crystal 10 may include machining a hole 20 in the second layer 14. The hole 20 may be the same as the previously described hole 20. The hole 20 may be machined using various techniques that can produce holes 20 within the second layer 14 on a micro-scale. For example, the method of manufacturing a photonic crystal 10 may include machining a hole 20 in the second layer 14 with a laser 18. The laser 18 may machine the hole 20, such as a plurality of holes 20, in one pulse. Alternatively, the laser 18 may machine the hole 20, such as a plurality of holes 20, with multiple pulses. In such an example, the laser 18 may be turned on at regular or irregular intervals to provide multiple pulses. In some non-limiting embodiments, the laser 18 may be an excimer laser. In some non-limiting embodiments, the pulse duration of the laser 18 may be, but is not limited to, 1 femtosecond to 10 microseconds. In some non-limiting embodiments, the laser 18, such as an excimer laser, may have a wavelength of about 190 nm. Alternatively, the laser 18, such as an excimer laser, may have a different wavelength. In some non-limiting embodiments, the laser 18 can provide a continuous laser beam to machine the hole 20, such as a plurality of holes 20.

In some non-limiting embodiments, the machining a hole 20 in the second layer 14 may include machining the hole 14 with a first diameter 22a from an outer surface 30 of the second layer 14 to a first hole depth 24a. The first diameter 22a and first hole depth 24a may be the same as the previously described first diameter 22a and first hole depth 24a. In some non-limiting embodiments, the machining a hole 20 in the second layer 14 may include machining the hole 20 with a second diameter 22b from the first hole depth 24a to a second hole depth 24b. The second diameter 22b and second hole depth 24b may be the same as the previously described second diameter 22b and second hole depth 24b. In such an example, the first diameter 22a may be larger than the second diameter 22b. In some non-limiting embodiments, the machining a hole 20 in the second layer 14 may include machining the hole 20 with a third diameter 22c from the second hole depth 24b to a third hole depth 24c. The third diameter 22c and the third hole depth 24c may be the same as the previously described third diameter 22c and third hole depth 24c. In such an example, the second diameter 22b may be larger than the third diameter 22c.

Providing a hole 20 or holes 20 with multiple diameters may generate multiple resonance peaks in the reflection/transmitted spectrum which further overlap with the resonance peak due to a pathogen (or antibody), which may lead to higher Q-factor resonance detection resulting in enhanced sensitivity. Similarly, providing holes 20 with varied depths in a photonic crystal 10 may generate multiple modes of light modulation, which may result in several resonance peaks of detection and can be optimized by the highest reflection/transmission peaks.

In some non-limiting embodiments, a third layer 16 may be present under the first layer 12. The third layer 16 may be the same as the previously described third layer 16. In such an example, the method of manufacturing a photonic crystal 10 may include: providing a third layer 16, depositing the first layer 12 over the third layer 16, and depositing the second layer 14 over the first layer 12.

In some non-limiting embodiments, the method of manufacturing a photonic crystal 10 may include depositing an antibody or antibodies into the hole 20. The antibody or antibodies may be any of the previously described antibody or antibodies. Alternatively, the method of manufacturing a photonic crystal 10 may include depositing a pathogen into the hole 20 instead of an antibody. The pathogen may be any of the previously described pathogens. In some non-limiting embodiments, the method of manufacturing a photonic crystal 10 may include depositing a linking molecule into the hole 20. The linking molecule may be any of the previously described linking molecules. The linking molecule may aid in bonding the antibody or pathogen depositing into the hole 20 to the photonic crystal 10, such as the second layer 14 and/or the first layer 12 of the photonic crystal 10.

In some non-limiting embodiments, the method of manufacturing a photonic crystal 10 may include machining a plurality of holes 20 in the second layer 14. The plurality of holes 20 may be the same as the previously described hole 20. In some non-limiting embodiments, each hole 20 of the plurality of holes 20 may be different. For example, each hole 20 of the plurality of holes 20 may have a different first diameter 22a, second diameter 22b, third diameter 22c, first hole depth 24a, second hole depth 24b, and/or third hole depth 24c. In such an example, each hole 20 of the plurality of holes 20 may independently have a different first diameter 22a, second diameter 22b, third diameter 22c, first hole depth 24a, second hole depth 24b, and/or third hole depth 24c as previously described. Machining holes 20 with varying depths and/or with different diameters will allow multiple peaks in the reflected or transmitted spectrum, which may lead to the detection of pathogens with a high-Q resonance; resulting in high sensitivity, high accuracy, and a lower limit of detection.

In some non-limiting embodiments, the laser 18, such as an excimer laser, may be configured to machine the photonic crystal 10 with a plurality of holes 20 in less than 1 hour, or less than 30 minutes, or less than 20 minutes, or less than 10 minutes.

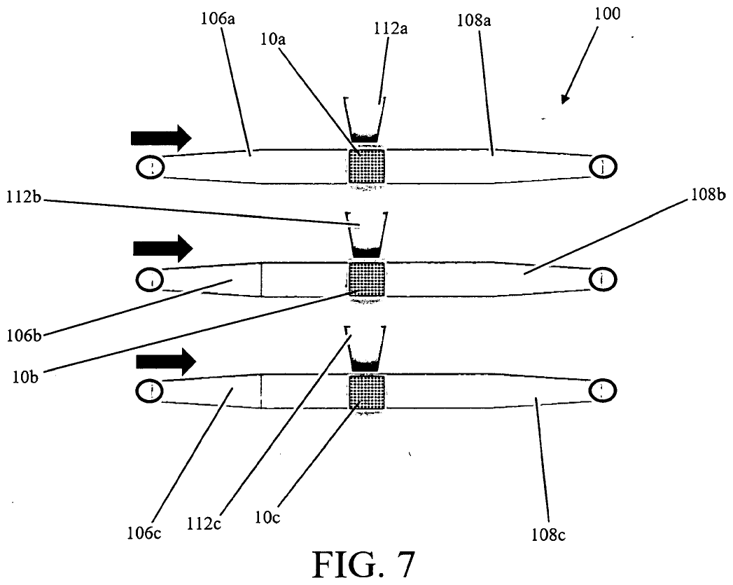

Referring to FIGS. 6A-7, the present disclosure also relates to a detection device 100 for detecting pathogens in a fluid sample. In some non-limiting embodiments, the detection device 100 may have a length between 0.1 cm and 100 cm, a width between 0.1 cm and 100 cm, and a thickness between 1 mm and 1,000 mm.

In some non-limiting embodiments, the detection device 100 may comprise a substrate 102. The substrate 102 may include various materials, such as, but not limited to, pyrex glass (i.e., borosilicate glass), plate glass, crown glass, quartz glass, polyethylene terephthalate (PET), sapphire, polytetrafluoroethylene (i.e., Teflon). For example, the substrate 102 may include pyrex glass (i.e., borosilicate glass). In such an example, the substrate 102 may have a refractive index of less than 2, such as, for example, about 1.47.

In some non-limiting embodiments, the detection device 100 may include a device body 104. The device body 104 may be positioned over the substrate 102. In some non-limiting embodiments, the device body 104 may include a material including polymers and/or glass. Non limiting examples of materials for the device body 104 include, but are not limited to, polydimethylsiloxane (PDMS), soda-lime glass, polymethyl methacrylate (PMMA), and sapphire. In some non-limiting embodiments, the device body 104 may include an inlet channel 106. The inlet channel 106 may be configured to transport a fluid sample to a photonic crystal 10. In some-non-limiting embodiments, the device body 104 may include an outlet channel 108. The outlet channel 108 may be configured to transport a fluid sample from a photonic crystal 10.

In some non-limiting embodiments, the detection device 100 may include a photonic crystal 10. The photonic crystal 10 may be the previously described photonic crystal 10. In some non-limiting embodiments, the photonic crystal 10 may be positioned between the substrate 102 and the device body 104. The photonic crystal 10 may include a detection area 116. In some non-limiting embodiments, the device body 104 may include a cutout portion at the detection area 116 of the photonic crystal 10 such that the device body 104 does not cover the detection area 116. The inlet channel 106 may be in fluid communication with the detection area 116 of the photonic crystal 10 and configured to transport a fluid sample to the detection area 116 of the photonic crystal 10. The outlet channel 108 may be in fluid communication with the detection area 116 of the photonic crystal 10 and configured to receive the fluid sample from the detection area 116 of the photonic crystal 10. The fluid sample may include various bodily fluids including, but not limited to, blood, serum, saliva, and/or the like. The detection area 116 refers to an area of the photonic crystal 10 that comprises an hole 20, such as a plurality of holes 20, in which an antibody or pathogen has been positioned in. This detection area 116 is configured to receive a fluid sample where pathogens or antibodies in the fluid sample bond to the antibodies or pathogens positioned within the hole(s) 20. The detection area 116 of the photonic crystal 10 (i.e., the area of the photonic crystal 10 configured to receive a fluid sample) may have a size of between 100 μm and 10 mm in one direction, and between 100 μm and 10 mm in another direction.

In some non-limiting embodiments, the photonic crystal 10 may include a plurality of holes 20 as previously described. Providing a plurality of holes 20 on a photonic crystal 10 may allow the refractive index to be varied periodically within the plane of the photonic crystal 10. In such an example, light 112 from a light source that passes through or is reflected from the photonic crystal 10 can exhibit a resonance wavelength. The resonance wavelength can be tuned by changing the angle of incidence, periodicity and height of the light source relative to the photonic crystal 10. In some non-limiting embodiments, the resonance wavelength can change in the presence or absence of pathogens attached to the antibody on the photonic crystal 10, or in the presence or absence of antibodies attached to the pathogen on the photonic crystal 10.

In some non-limiting embodiments, the detection device 100 may include a light source. The light source may be configured to expose the detection area 116 of the photonic crystal 10 to light 112 from the light source. The light 112 provided from the light source may have a wavelength between 400 nm and 500 μm. In some non-limiting embodiments, the light 112 from the light source may be monochromatic light. In some non-limiting embodiments, the light 112 from the light source may be transmitted through the photonic crystal 10 as transmitted light 114b. In some non-limiting embodiments, the light 112 from the light source may be reflected from the photonic crystal 10 as reflected light 114a. In some non-limiting embodiments, a portion of the light 112 from the light source may be transmitted through the photonic crystal 10 as transmitted light 114a and another portion of the light 112 from the light source may be reflected from the photonic crystal 10 as reflected light 114a. The transmitted light 114b and/or the reflected light 114a may have a wavelength between 400 nm and 500 μm.

In some non-limiting embodiments, the detection device 100 may include a light detector. The light detector may receive the transmitted light 114b and/or reflected light 114a from the photonic crystal 10 and analyze various optical properties of the received light to determine if antibodies and/or pathogens are present in the fluid sample, as is known in the art for photonic crystals 10.

In some non-limiting embodiments, the light source and the light detector may include the same device. For example, a Fourier-transform infrared spectroscopy (FTIR) apparatus may be used as both the light source and the light detector. In such an example, the FTIR apparatus may expose the photonic crystal 10 to light 112 and then receive the transmitted light 114b and/or reflected light 114a from the photonic crystal 10 for analysis. In some non-limiting embodiments, the light source and/or light detector may be a handheld FTIR apparatus. In some non-limiting embodiments, the light source and/or light detector may be a near IR optical reader.

Referring to FIG. 7, in some non-limiting embodiments, the detection device 100 may include a plurality of photonic crystals 10a, 10b, 10c, each of which are configured to receive the fluid sample. In such an example, each photonic crystal may include a different antibody or pathogen and/or linking molecules, which allows for the detection of multiple pathogens or antibodies at one time. Each photonic crystal of the plurality of photonic crystals 10a, 10b, 10c may be in fluid communication with the inlet channel 106 and the outlet channel 108. Alternatively, each photonic crystal of the plurality of photonic crystals 10a, 10b, 10c may receive the fluid sample from a different inlet channel and a different outlet channel receives the fluid sample from the photonic crystal. For example, a first photonic crystal 10a may be in fluid communication with a first inlet channel 106a and a first outlet channel 108a, a second photonic crystal 10b may be in fluid communication with a second inlet channel 106b and a second outlet channel 108b, and a third photonic crystal 10c may be in fluid communication with a third inlet channel 106c and a third outlet channel 108c. Any number of photonic crystals 10 may be included in the detection device 10 and the number of photonic crystals 10 chosen may be based on the number of pathogens or antibodies to be detected. In some non-limiting embodiments, a light source may provide light 112 to each photonic crystal of the plurality of photonic crystals 10a, 10b, 10c. Alternatively, a first light source may provide light 112a to a first photonic crystal 10a, a second light source may provide light 112b to a second photonic crystal 10b, and a third light source may provide light 112c to a third photonic crystal 10c. A detection device 100 that includes a plurality of photonic crystals 10 has the benefit of allowing the detection of a large number of pathogens or antibodies at the same time.

In some non-limiting embodiments, the detection device 100 may be configured to detect pathogens and/or antibodies in a fluid sample in less than 1 hour, or less than 30 minutes, or less than 20 minutes, or less than 10 minutes, or less than 5 minutes, or less than 2 minutes.

The present invention is also directed to a method of detecting a pathogen in a fluid sample. In some non-limiting embodiments, the method may include providing a detection device 100. The detection device 100 may be the same as the previously described detection device 100. In some non-limiting embodiments, the detection device 100 includes a photonic crystal 10 as previously described. In some non-limiting embodiments, the photonic crystal 10 may include a hole 20, such as a plurality of holes 20. In some non-limiting embodiments, the detection device 100 may include a plurality of photonic crystals 10a, 10b, 10c. For example, the detection device 100 may include a plurality of photonic crystals 10a, 10b, 10c between the inlet channel 106 and the outlet channel 108. In some non-limiting embodiments, the method may include introducing a fluid sample into an inlet channel 106. For example, the fluid sample may be a bodily fluid. Examples of bodily fluids that may be used in the detection device include, but are not limited to, blood, serum, saliva, and/or the like.

In some non-limiting embodiments, the method may include passing the fluid sample from the inlet channel 106 to the photonic crystal 10. In such an example, the fluid sample is passed over the detection area 116 of the photonic crystal 10. In some non-limiting embodiments, the fluid sample that is passed over the detection area 116 of the photonic crystal 10 may flow into the hole 20, such as the plurality of holes 20. When the fluid sample flows into the hole(s) 20, pathogens or antibodies present in the fluid sample may bond to the antibody or pathogen that is present in the hole(s) 20.

In some non-limiting embodiments, after the fluid sample has been passed over the photonic crystal 10, the method may include exposing the fluid sample and the photonic crystal 10 to light 112 from a light source. The light 112 from the light source may be the same as the light 112 and the light source previously described. For example, the light 112 from the light source may be monochromatic light having a wavelength between 380 nm and 100,000 nm. In some non-limiting embodiments, the method may include detecting the light that passes through (i.e., transmitted light 114b) and/or is reflected from (i.e., reflected light 114a) the photonic crystal 10 with a light detector. The transmitted light 114b, reflected light 114a, and the light detector may be the same as previously described. For example, the light source and the light detector may be the same device, such as an FTIR spectroscopy apparatus as previously described.

In some non-limiting embodiments, after the fluid sample has been passed over the photonic crystal 10, the method may include passing the fluid sample to the outlet channel 108.

The present invention has been described with reference to certain exemplary embodiments, dispersible compositions and uses thereof. However, it will be recognized by those of ordinary skill in the art that various substitutions, modifications or combinations of any of the exemplary embodiments may be made without departing from the spirit and scope of the invention.

1. A photonic crystal for detection of an analyte, comprising:
a first layer comprising a first material with a first refractive index;
a second layer over the first layer and comprising a second material with a second refractive index that is higher than the first refractive index;
wherein the second layer comprises a hole, the hole comprising:
a first diameter from an outer surface of the second layer to a first hole depth;
a second diameter from the first hole depth to a second hole depth;
wherein the first diameter is larger than the second diameter; and
a member of a binding pair with the analyte linked to a surface of the hole.

2. The photonic crystal of claim 1, wherein the first material comprises silicon dioxide, nitride, indium phosphide, lithium niobite, sapphire, or a combination thereof.

3. (canceled)

4. The photonic crystal of claim 1, further comprising a third layer under the first layer and comprising a third material with a third refractive index, wherein the third refractive index is higher than the first refractive index.

5. The photonic crystal of claim 4, wherein the second material and the third material each independently comprise silicon, gallium arsenide, gallium nitride, silicon carbide, indium gallium arsenide, or a combination thereof.

6. The photonic crystal of claim 5, wherein the second material and the third material comprise silicon.

7. The photonic crystal of claim 1, wherein the second layer comprises a plurality of holes.

8. The photonic crystal of claim 1, wherein the hole comprises a third diameter from the second hole depth to a third hole depth, wherein the second diameter is larger than the third diameter.

9. The photonic crystal of claim 1, wherein the first diameter is between 0.5 μm and 5 μm and/or the second diameter between 0.25 μm and 4 μm.

10. (canceled)

11. The photonic crystal of claim 1, wherein the first hole depth is between 0.1 μm and 1 μm and/or the second hole depth in between 0.25 μm and 2 μm or the thickness of the second layer.

12. (canceled)

13. (canceled)

14. The photonic crystal of claim 8, wherein the third diameter is between 0.1 μm and 3 μm and/or the third hole depth is between 0.5 μm to 3 μm or the thickness of second layer.

15. (canceled)

16. (canceled)

17. The photonic crystal of claim 1, wherein the member of a binding pair with the analyte is an antibody.

18. The photonic crystal of claim 1, wherein the analyte is a pathogen or an antibody specific to a pathogen.

19. The photonic crystal of claim 1, comprising one or more additional holes and the holes are discretely-addressable in an array, wherein at least one of the one or more additional holes comprises a different member of a binding pair as compared to the member of a binding pair with the analyte bound to its surface, or no member of a binding pair bound to its surface.

20. A method of manufacturing a photonic crystal, comprising:
providing a first layer comprising a first material with a first refractive index;
depositing a second layer comprising a second material with a second refractive index that is higher than the first refractive index over the first layer;
machining a hole in the second layer with a laser, comprising:
machining the hole with a first diameter from an outer surface of the second layer to a first hole depth;
machining the hole with a second diameter from the first hole depth to a second hole depth;
wherein the first diameter is larger than the second diameter; and
linking a member of a binding pair with an analyte linked to a surface of the hole.

21. The method of claim 20, wherein the laser is an excimer laser.

22. The method of claim 20, wherein the machining of the hole further comprises: machining the hole with a third diameter from the second hole depth to a third hole depth, wherein the second diameter is larger than the third diameter.

23. The method of claim 20, further comprising: depositing an antibody or antibodies into the hole.

24. The method of claim 20, further comprising machining a plurality of holes in the second layer.

25. A method of detecting pathogens in a fluid sample, such as blood, serum, saliva, and/or another bodily fluid of a patient, comprising:
providing a detection device comprising:
a substrate;
a device body positioned over the substrate and comprising an inlet channel and an outlet channel;
a photonic crystal in fluid communication with the inlet channel and the outlet channel, the photonic crystal comprising:
a first layer comprising a first material with a first refractive index;
a second layer over the first layer and comprising a second material with a second refractive index that is higher than the first refractive index;
wherein the second layer comprises a hole, the hole comprising:
a first diameter from an outer surface of the second layer to a first hole depth; and
a second diameter from the first hole depth to a second hole depth;
wherein the first diameter is larger than the second diameter; and
a member of a binding pair with the analyte linked to a surface of the hole;
introducing the fluid sample into the inlet channel;
passing the fluid sample over the photonic crystal;
exposing the fluid sample and photonic crystal to light from a light source;
detecting the light that passes through and/or is reflected from the photonic crystal with a light detector; and
passing the fluid sample to the outlet channel.

26. (canceled)

27. (canceled)

28. The method of claim 25, wherein the light source and the light detector is a Fourier-transform infrared spectroscopy device.

29. The method of claim 25, wherein the photonic crystal comprises a plurality of holes.

30. The method of claim 25, further comprising a plurality of photonic crystals between the inlet channel and the outlet channel, wherein each photonic crystal of the plurality of photonic crystals comprises a different antibody or antibodies.
